(19) United States
(12) Patent Application Publication
(10) Pub. No.: US 2019/0365338 A1
(43) Pub. Date: Dec. 5, 2019

(54) METHOD FOR MONITORING THE STATUS OF AN X-RAY TUBE ASSEMBLY FOR AN X-RAY FACILITY THAT INCLUDES AN X-RAY SOURCE, AN X-RAY TUBE ASSEMBLY, A COMPUTER PROGRAM, AND AN ELECTRONICALLY READABLE DATA CARRIER

(71) Applicant: Siemens Healthcare GmbH, Erlangen (DE)

(72) Inventors: Sultan HAIDER, Erlangen (DE); Joerg FREUDENBERGER, Kalchreuth (DE)

(73) Assignee: Siemens Healthcare GmbH, Erlangen (DE)

(21) Appl. No.: 16/419,066

(22) Filed: May 22, 2019

(30) Foreign Application Priority Data
May 29, 2018 (EP) 18174897.1

(51) Int. Cl. A61B 6/00

(52) U.S. Cl. CPC A61B 6/461

(57) ABSTRACT

A method is for monitoring the status of an X-ray tube assembly for an X-ray facility, including an X-ray source. In an embodiment, the method includes determining input information describing at least one of an application of the X-ray source, a current status of the X-ray source, and a current status of surroundings of the X-ray source. The method further includes deriving status information to be issued to a user, using the input information determined; and displaying the status information derived on an indicator device. The condition-related information, describing transportation conditions during transportation, is used as the input information.

### PRIORITY STATEMENT

The present application hereby claims priority under 35 U.S.C. § 119 to European patent application number EP 18174897.1 filed May 29, 2018, the entire contents of each of which are hereby incorporated herein by reference.

### FIELD

Embodiments of the invention generally relate to a method for monitoring the status of an X-ray tube assembly for an X-ray facility, which assembly includes an X-ray source. Embodiments of the invention further generally relate to an X-ray tube assembly, a computer program and an electronically readable data carrier.

### BACKGROUND

X-ray facilities of various types are used in medical imaging as an established imaging modality. For example, C-arm X-ray facilities, computed tomography facilities and other types of X-ray facilities are known. Each X-ray facility comprises an X-ray source, such as an X-ray tube, and an X-ray detector in order to acquire X-ray images using the X-rays that pass through the imaging region in the patient, and which are generated by the X-ray source.

Modern X-ray sources, which together with a housing and/or other fastening device(s) form an X-ray tube assembly (or X-ray tube device), are sensitive facilities and at the same time parts of the X-ray facility that are subject to wear, that is, an X-ray source has a specific life for which it can be used in the X-ray facility. Even an increase in the temperature of the X-ray source during its operation and also other effects can lead to limitations in its application, such as limiting the period of use in a certain type of examination. This also includes the operating parameters of the X-ray source, for example, a tube voltage in an X-ray tube used as an X-ray source.

Therefore, the user of the X-ray tube assembly is confronted with a wide range of questions during the use of the X-ray source, with regard, for example, to being able to determine the status of the X-ray source, in particular, regarding for how long it can be used in a certain type of examinations with a specific frequency, how the X-ray tube facility is to be operated for maximum quality, how maintenance measures can be carried out, for example, replacing the X-ray tube and the entire X-ray assembly and such like. Since the X-ray source is a sensitive technical device, in addition particular caution is required in the transportation thereof.

Nowadays, such information is usually transmitted to the relevant service engineers or other system users via handbooks or training courses, which is expensive and time-consuming. Information about the status of the X-ray source and support during the use of the X-ray source, in particular an examination, is not nowadays available on the X-ray source or X-ray assembly.

For many aspects of the status of the X-ray sources, the use of data-mining tools, which derive predictions on future use from specific parameters of the X-ray source and can make them available to users, has been suggested. In such data-mining approaches, however, contracts between manufacturers and users are generally required.

### SUMMARY

In addition, it is possible to derive status information relating to an X-ray source indirectly through specific service-software applications of the X-ray facility, in which the X-ray tube assembly is used. Even so, the inventors have discovered that the necessary information is then more likely to be scattered and an extremely complex software application is involved, such that obtaining status information about the X-ray source represents a considerable effort for a user. On the X-ray tube assembly itself, in most cases all that is available are stickers, which may contain object and manufacturer information about the X-ray source, for example, the manufacturer, a serial number, technical specifications and suchlike. The inventors have discovered that no information is therefore available on the current status, that is, the status of the X-ray source.

At least one embodiment of the invention is therefore directed to providing a possibility for improved user information relating to the status of the X-ray source.

Embodiments of the invention are directed to a method, an X-ray tube assembly, a computer program, and an electronically readable data carrier.

A method according to at least one embodiment of the invention is for monitoring the status of an X-ray tube assembly for an X-ray facility that includes an X-ray source, the method comprising:

determining input information describing the application of the X-ray source and/or the current status of the X-ray source and/or its surroundings by at least one determination device(s),

deriving status information to be issued to a user, using the input information via a processing device(s), and

displaying the status information on an indicator device, in particular on a display.

Alongside the method, at least one embodiment of the present invention also relates to an X-ray tube assembly, comprising

an X-ray source,

a control unit with a determination device(s) for determining input information describing the application of the X-ray source and/or the current status of the X-ray source and/or the environment thereof and a processing device(s) for deriving status information to be issued to a user, utilizing the input information, and

an indicator device that can be activated by the control unit, for displaying the status information.

At least one embodiment of the present invention also relates to an X-ray tube assembly, comprising:

an X-ray source;

at least one processor, configured to determine input information, the input information describing at least one of an application of the X-ray source, a current status of the X-ray source, and surroundings of the X-ray source, and configured to derive status information to be issued to a user, utilizing the input information determined; and an indicator device, controllable by the at least one processor, to display the status information derived, wherein condition-related information, describing transportation conditions during transportation, is used as the input information.

All the statements relating to the method according to embodiments of the invention can be applied by analogy to the X-ray tube assembly with integrated status monitoring system according to embodiments of the invention.

A computer program according to at least one embodiment of the invention is, for example, loadable directly into a memory of a control facility and comprises programming code/segments/modules to carry out the steps of the method described herein when the program is run in the control facility. The computer program to which, of course, the statements relating to the method likewise apply, can be stored on an electronically readable data carrier according to at least one embodiment of the invention, which therefore includes electronically readable control information stored thereon, which includes at least one aforementioned computer program and is embodied such that, when the data carrier is used in a control facility, it carries out a method described herein. The computer program can be embodied as an app for mobile devices.

### BRIEF DESCRIPTION OF THE DRAWINGS

Further advantages and details of the present invention will emerge from the example embodiments described hereinafter and from the drawings, in which:

FIG. 1 shows a flow chart showing the principles of the method according to an embodiment of the invention,

FIG. 2 shows the design of an X-ray tube assembly, and

FIG. 3 shows an external view of an X-ray tube assembly.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Most of the aforementioned components, in particular the identification unit, can be implemented in full or in part in the form of software modules in a processor of a suitable control device or of a processing system. An implementation largely in software has the advantage that even control devices and/or processing systems already in use can be easily upgraded by a software update in order to work in the manner according to at least one embodiment of the invention.

A method according to at least one embodiment of the invention is for monitoring the status of an X-ray tube assembly for an X-ray facility that includes an X-ray source, the method comprising:

determining input information describing the application of the X-ray source and/or the current status of the X-ray source and/or its surroundings by at least one determination device(s),

deriving status information to be issued to a user, using the input information via a processing device(s), and

displaying the status information on an indicator device, in particular on a display.

It is therefore suggested, in an embodiment, to use an indicator device, in particular a display, dedicated to issuing status information relating to the X-ray tube assembly and consequently assigning it to the X-ray tube assembly with its X-ray source, at least on a temporary basis. Here, the indicator device is particularly advantageously permanently assigned to the X-ray source, and therefore, in particular, even forms part of the X-ray tube assembly. This means that in a particularly advantageous manner an indicator device, in particular a display, can be used that is affixed in, on, or adjacent to the X-ray assembly and/or pertaining to said assembly.

As an alternative, or in addition to this, other embodiments are available in which, for example, external indicator devices are used, be it for example, indicator devices that are provided in any case on the X-ray facility or, for example, involving an appropriate software application, responsive display facilities on a user's mobile devices, such as a smartphone, for example. In this case, a computer program for implementing an embodiment of the method can include, for example, an application (an “app”) for the mobile device that can establish communications links to sources of input information, for example to a control facility of the X-ray facility and/or to an external server facility, with the downloadable software application itself then also being able to constitute the determination device and the processing device.

Of course, it is also possible for there to be spatially separated embodiments of the method according to embodiments of the invention and different styles of implementation, for example through a webpage portal, which communicates, for example, with the X-ray facility and/or with an external server facility on which the input information is provided, and processes this accordingly. Obviously various specific embodiments are provided to implement such a “status display”, with, as already mentioned, the X-ray tube assemblies being extended in the preferred embodiment to include an indicator device, in particular a display, and also a processing device(s) and the at least one acquisition device(s). Here, an active or preferably even an interactive display is added to the X-ray source, which until now was a passive component.

As a result of the new options, a wide range of advantages open up for the manufacturer using the method according to the invention, in particular, by creating more user-friendly X-ray sources, in particular X-ray tubes, and the points in the interaction with the user that were previously problematic regarding information relating thereto can be targeted.

To be specific, here at least some of the input information can be retrieved by the control facility in the X-ray facility and/or by a control unit (such as at least one processor, for example) of the X-ray tube assembly and/or an external server facility and/or at least partly acquired by at least one of determination device(s) assigned to the X-ray tube assembly or determination device(s) pertaining thereto, in particular a sensor. Some of the useful input information is already acquired in modern X-ray facilities in some embodiments, such that this information that has been acquired accordingly thereon can be usefully accessed in the control facility of the X-ray facility. In a particularly advantageous manner, in particular for determining input information relating to the current operating status or general status of the X-ray source such as its temperature and suchlike, sensors or other determination device(s) are used, which preferably perform their acquisition operations directly on the X-ray source or the X-ray tube assembly.

The following can be acquired as input information: application information describing the application of the X-ray source in the past and/or for an immediately imminent measurement and/or age information describing the age of the X-ray source and/or conditions-related information describing storage conditions and/or transportation conditions. Here, conditions-related information can of course also relate to operating conditions and, for example, to a temperature of the X-ray source and/or a humidity of the environment of the X-ray source and/or vibrations induced into the X-ray tube assembly and/or an oil pressure in the X-ray source can be used.

It is specifically the use of conditions-related information as input information that also allows in particular transportation monitoring of the X-ray source via the method according to an embodiment of the invention. In this way, specific provision can be made for transportation monitoring information to be acquired as status information when there is a transportation procedure of the X-ray tube assembly. As has already been mentioned, an X-ray source such as an X-ray tube is a sensitive technical device, the life and/or readiness for use of which may be highly dependent on the external conditions to which it is exposed.

For example, there are known to be X-ray tubes that should ideally be kept in a certain temperature range or in a specific humidity range. Moreover, vibrations should where necessary be kept within a permitted range. Such target ranges can be described by target values, such that it is particularly advantageous if the acquisition of the transportation monitoring function includes the comparison of input information with target values for transportation. This makes it possible in particular, in the case of transportation conditions outside the target range described by target values, to issue the status information as a warning message.

Therefore, specific embodiments are conceivable in which the indicator device, for example, continually shows the transportation monitoring information in green for example, while the X-ray tube assembly is being transported together with the X-ray source, whereas, when the input information displays values that fall outside the target ranges, a more sustained warning can be issued, in particular with additional acoustic and/or haptic and/or optical emission of a warning signal. It should be pointed out here that transportation monitoring can be incorporated as an indicator device in a particularly advantageous manner on a display pertaining to the X-ray tube assembly, which display is positionable within the field of vision of the person carrying out the transportation, in particular even during the transportation of the X-ray tube assembly or is positionable visible to said person and suchlike.

Moreover, in an embodiment it is also useful to keep records of transportation procedures, for example, by storing the transportation monitoring information or at least logging warning events during a transportation procedure and, for example, storing them in a memory of the processing device(s) or in a control unit that incorporates the processing device(s) (such as at least one processor, for example) in order to be retrieved therefrom later. If there is a communications link in any case between the status monitoring system described here and an external server facility, for instance to the internet, of course warnings and suchlike generated in a transportation procedure will therefore also be transmitted directly to the external server facility, logged thereon, and/or evaluated for the emission of a local alarm.

The observation should also be made at this point that not only can the status information be usefully made accessible during transportation, but in particular when using various display devices and/or various status information, useful status information can also be issued to a user in further operational states of the X-ray source, for example, during the installation, during the calibration, during application, in cases of faults, during maintenance procedures, while uninstalling and/or during a refurbishment.

Therefore, depending on a current application phase, different status information can be provided, it being possible in the case of an interactive display device, which therefore includes an input device or to which an input device is assigned, for a user input also to have an influence on the status information that is actually shown, since for example, a user can select a user phase, for example “transport”, “installation”, “calibration”, and suchlike.

Alongside the example of transportation monitoring information, a plurality of other useful status information can be generated and issued.

A particularly advantageous further development of an embodiment of the present invention makes provision for prediction information that describes the future usability of the X-ray source to be acquired as status information. Such prediction information can, for example, be an expected future life span, which can be determined as a function of data about the past application of the X-ray source, for example, examinations that have been carried out, the number of examinations that have been carried out or type of examinations carried out and such like. Also, information about examinations currently to be carried out, for instance a current measurement protocol, can be entered, it being possible for wear information relating to the X-ray source to be made available to the processing device(s), for example, also as input information, the basis of which wear information can also be, for example, previous examinations relating to the life and/or properties relating to the X-ray source or to a comparable X-ray source that is in use.

For status monitoring during installation, it is possible, for example, for information about plugged in connections to be included in the status information. During the use thereof for imaging, it is possible, for example, for current operating parameters of the X-ray source to be displayed, just like information relating to the heat management of the X-ray source, which proves to be useful in particular for examinations that continue for longer. This is expedient in particular when the X-ray source is not conspicuously installed, and the temperature thereof cannot be observed directly and suchlike.

Expediently, the status information can be issued as a function of a user input via an input device that is assigned to the indicator device and/or to the X-ray tube assembly and/or to the X-ray facility and/or incorporated in one unit with the indicator device and/or issued as a function of the fulfillment of an output condition that in particular evaluates at least one item of input information. This means that status information can be requested, for example, by relevant user inputs via an input device, it also being possible, however, by fulfilling output conditions as a triggering event, for example, to issue automatically at appropriate time intervals status information that a user might need at this time. Thus, it is possible automatically, for example, to begin issuing the current operating parameters and/or information about heat management as status information, for example, at the start of X-ray generation by the X-ray source, which information can be displayed until the end of the current operating phase. For example, if an error state occurs, a corresponding output condition can ensure that the indicator device is activated where necessary and issues information about the currently prevalent error as status information. Via the user inputs, a type of menu can also be created, for example, in which a user can alternate between different types of status information.

It is particularly expedient, in addition to and/or subsequent to a user input and/or fulfillment of a further output condition, to issue on the indicator device at least one further item of output information relating to the X-ray source, in particular likewise acquired as a function of the input information. This means that, apart from status information, the indicator device can also forward to a user further useful output information relating to the X-ray source. For example, it is possible for maintenance information describing a maintenance procedure and/or handbook information and/or object information describing a technical feature and/or designation of the X-ray source and/or manufacturer-related information, in particular a logo, describing the manufacturer of the X-ray source, and/or examination-related information relating to an examination currently to be carried out using the X-ray source and/or educational information explaining the operation of the X-ray source and/or of the X-ray facility to be used as output information.

It is precisely in connection with status information that maintenance information is particularly appropriate output information, since for example, maintenance steps can then be explained and conveyed to a user via the indicator device. In particular in the case of indicator devices spatially assigned to the X-ray tube assembly, the user can then work, for example, at an X-ray tube assembly and obtain useful information in a direct context via the indicator device. Where an input device, for example an appropriate screen, is used in addition, the user can, for example, be guided step by step through a maintenance process, for example, through the replacement of components, with the corresponding maintenance information being preferably complemented accordingly with relevant, useful status information, for example, pointing out open maintenance flaps, connection ports available, current error states and suchlike.

It is also possible for object information to be displayed on the indicator device, for example, the type of X-ray source, technical specifications of the X-ray source, the name of the manufacturer, the place of manufacture, the serial number, a logo or other object-/manufacturer-related information. A logo indicating the manufacturer or type of X-ray source can also be used as a kind of screensaver if no status information of a different kind is in fact being displayed.

If the X-ray source is in fact being used for an examination, examination-related information pertaining thereto can be issued, for example, a body region that is to be examined, a measurement protocol that has been selected, an examination duration, information about other components of the X-ray facility, information about the patient and suchlike.

In a particularly useful embodiment, the status monitoring system can also have an educational mode of operation in which the indicator device is used to issue educational information regarding the operation of the X-ray source and/or the X-ray facility. Such a teaching mode of operation informs/teaches the users about a wide range of factors connected with the X-ray source, for example, X-ray physics, operating parameters of the X-ray source, functionalities of the X-ray source, application profiles and suchlike.

As already indicated, it can be expedient in the context of the present invention to use a plurality of indicator devices, on which in particular various status information is used and/or operated at different time intervals. For example, an indicator device and/or status information can be assigned in each case to application phases including transport, installation, calibration, imaging, maintenance, expansion and suchlike.

It is particularly preferable in the context of at least one embodiment of the present invention, as already mentioned, to use an indicator device, in particular a display, that is affixed on or adjacent to the X-ray tube assembly and/or pertains thereto. For such an indicator device, what is known as “electronic paper” is available in particular. Here, for example, electrophoretic ink (also known as electronic ink) can be laminated onto a carrier, a plastic film for example, and then combined with appropriate electronics in order to create a display made from electronic paper. Such electronic paper is particularly suitable for installation, since in particular it can also be available in flexible form.

In the case of indicator devices provided on the X-ray tube assembly itself, a further useful embodiment makes provision for a machine-readable code which directs the user to a different information source, in particular in the internet, to be issued as status information and/or as further output information. The machine-readable code can in particular be a QR code, which moreover, can also be displayed advantageously using electronic paper. Such a machine-readable code can now be read, for example, by a user's mobile device and can be used to access the further information source.

For example, in a specific embodiment it is conceivable to issue a QR code that directs a service technician to information sources that are useful for his task, for example, web pages that display specific information, such as installation instructions, spare parts logistics, compatible generators and interfaces, contact data for second-stage support, and suchlike.

Moreover, in the context of the provision of the indicator device on the X-ray tube assembly, it is also preferable if at least some of the determination device(s) and/or of the processing device(s) (such as at least one processor, for example) also form part of the X-ray tube assembly, in particular as a control unit. In this way, the X-ray tube assembly is therefore provided with integrated intelligence, which provides useful information for the user that was not available in the passive X-ray sources that were known hitherto.

During the retrieval of at least some of the input information from a communications partner external to the determination device(s) and/or when using an indicator device external to the processing device(s), a wireless external communications link, in particular Bluetooth and/or NFC, can be used at least sometimes. In particular, this is also useful for control units (such as at least one processor, for example) that are provided on the X-ray tube assembly and that implement the determination device(s) and the processing device(s). Then a communications interface can be provided that does not have to be limited to near field communication such as Bluetooth and/or NFC but can also allow wireless communication with further removed server facilities, for example, server facilities on the internet. Examples of such more extensive interfaces include WLAN-interfaces and/or mobile radio interfaces. It is of course also possible, however, via communications standards such as Bluetooth and suchlike, to access the internet or other networks indirectly, for example via a control facility of the X-ray facility, in order to be able to retrieve the input information and/or be able to access external indicator devices.

An indicator device of a user's mobile device can also be used as an external indicator device that is preferably to be used additionally. For example, status information and/or further output information can also at least sometimes be issued on a user's mobile device, such as a smartphone; as already mentioned, it is also conceivable, however, for the status monitoring system to be completely implemented in the user's mobile device, for example, through a corresponding software application that incorporates the processing device(s) and the determination device(s).

At this point it should be pointed out once again that, even independently of mobile devices, touchscreens have proved to be suitable displays or indicator devices in the context of embodiments of the present invention.

In a development of at least one embodiment of the present invention, provision can also be made, where enabling data and a retrieval user input are available via an input device assigned to the indicator device, for at least one after-sales service provision to be consulted and output information relating to the after-sales service provision to be displayed on the indicator device. In particular when there is a connection to an external server facility, it can therefore also be facilitated in the context of at least one embodiment of the present invention for additional services to be provided if an appropriate contractual basis which is described by the enabling data is in place. Such additional services can include, for example, self-repair, failure predictions, refurbishment options and suchlike.

Alongside the method, at least one embodiment of the present invention also relates to an X-ray tube assembly, comprising

an X-ray source,

a control unit (such as at least one processor, for example) with a determination device(s) for determining input information describing the application of the X-ray source and/or the current status of the X-ray source and/or the environment thereof and a processing device(s) for deriving status information to be issued to a user, utilizing the input information, and

an indicator device that can be activated by the control unit, for displaying the status information.

All the statements relating to the method according to embodiments of the invention can be applied by analogy to the X-ray tube assembly with integrated status monitoring system according to embodiments of the invention.

A computer program according to at least one embodiment of the invention is, for example, loadable directly into a memory of a control facility and comprises programming code/segments/modules to carry out the steps of the method described herein when the program is run in the control facility. The computer program to which, of course, the statements relating to the method likewise apply, can be stored on an electronically readable data carrier according to at least one embodiment of the invention, which therefore includes electronically readable control information stored thereon, which includes at least one aforementioned computer program and is embodied such that, when the data carrier is used in a control facility, it carries out a method described herein. The computer program can be embodied as an app for mobile devices.

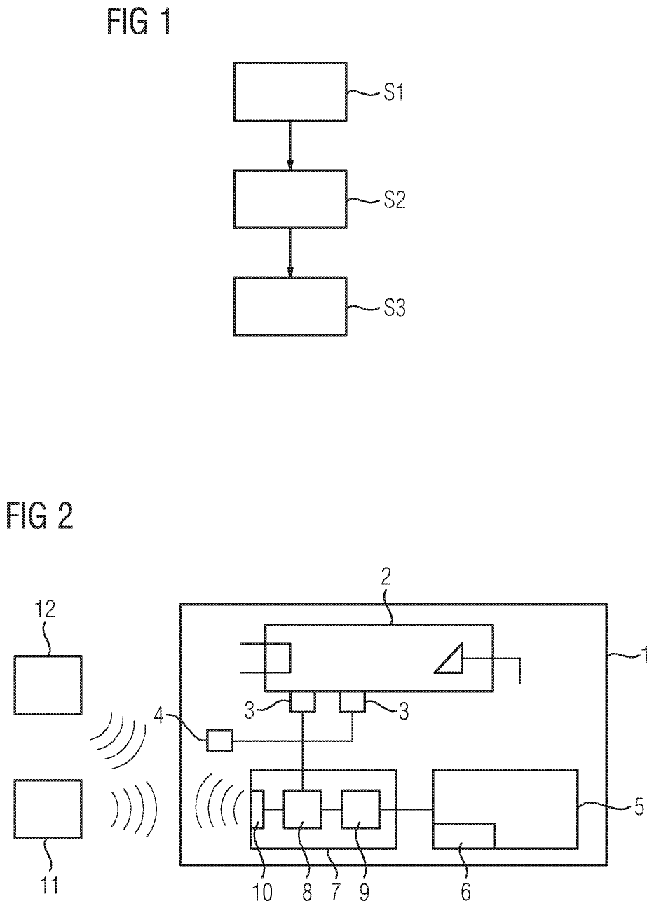

FIG. 1 illustrates the basic course of example embodiments of the method according to the invention. Here, an indicator device assigned to an X-ray tube assembly with an X-ray source, in particular a display, is used to issue status information. For this purpose, in step S1, at least one determination device(s) is used to acquire input information which relates to the X-ray source and is ultimately dependent in its type on the type of status information to be displayed, which is discussed in greater detail hereinafter. In the present case, the input information describes the application of the X-ray source and/or the current status of the X-ray source and/or the current status of the X-ray source and/or the current status of the environment of the X-ray source.

In step S2, status information to be issued to a user is derived from the input information using a processing device(s). This essentially means that the input information, which indeed describes physical-technical matters, is evaluated, taking into account technical factors relating to the X-ray source and optionally to the X-ray facility that is using said X-ray source in order to generate information that is useful for the user.

Then, in step S3, this status information is displayed on the indicator device, in particular on the display.

In preferred specific embodiments, different types of status information can be generated and displayed, in particular depending on an application phase of the X-ray source and/or on user inputs. Nevertheless, it should be pointed out that upstream of steps S1, S2 and S3, various output conditions can be checked before the relevant steps S1, S2, S3 are carried out. Here, output conditions can of course also evaluate user inputs on an input device assigned to the indicator device, in particular on an input device that is integrated therein, such as in the case of a touchscreen. For example, upstream of step S1, a check can be made as to whether the acquisition and issue of the status information is to currently ensue/is desirable/is sensible. This may depend, for example, on the application phase of the X-ray tube assembly or X-ray source such that, for example, if transportation monitoring is to take place, it is possible via a user input or other available information to verify whether a transportation procedure is impending and such like. For example, on the indicator device a menu can also be displayed, in which the user can alternate between different application phases, such as final assembly, transportation, installation, calibration, application for imaging, maintenance, refurbishment and suchlike, whereupon status information that differs from this selection can then be generated and displayed.

In embodiments in which the input information is also to be included in an output condition, a corresponding check can then ensue after step S1; however, output conditions can also still be checked after step S2, when for example, the extent to which the issue of the status information is useful is to be made dependent on the status information. For example, embodiments are conceivable in which warnings are to be issued on the indicator device only for specific contents of the status information.

The indicator device can also be used here for the issue of further output information alongside the status information, the further output information preferably likewise relating to the X-ray source or at least to the X-ray tube assembly. Such output information can include in particular maintenance information, object-related information, manufacturer-related information, educational information and/or referrals to further information sources.

Preferably, at least one display is affixed as an indicator device on the X-ray tube assembly itself, for example on a housing that comprises and supports the X-ray source, which is preferably an X-ray tube. The relevant indicator device then shows the status of the X-ray source and various, optionally adaptable options and/or operating modes for different types of users.

If in an example embodiment, transportation monitoring is to take place, for example, during a transportation of the X-ray tube assembly, an indicator device that is visible during the transportation is preferably used. Physical parameters such as temperature, humidity, vibrations and suchlike that have been determined by acquisition device(s) in the X-ray tube assembly itself, in particular sensors, are determined as input information and evaluated as to whether the values described by the input information fall within target ranges described by target values. If this is the case, the relevant status information can be simply issued; however, if values fall outside the target ranges, a warning signal can additionally be provided acoustically and/or optically and/or haptically and/or the status information can be reproduced in a more conspicuous manner.

A different specific and useful application is the prediction of the probable life and usability of the X-ray source, the prediction being able to use as input information application information describing the application of the X-ray source in the past and/or currently, it being additionally possible in the evaluation to consult wear information that was obtained, for example, from tests with X-ray sources of the same type. For example, it can then be issued as status information that the X-ray source can probably continue to be used for a certain type of examinations of a certain type or suchlike.

During an examination, the indicator device can also be used to also issue examination-related information relating to the current examination, for example, an appropriately selected measurement protocol, a part of the patient's body that has been selected and is to be imaged, and such like. Here it is particularly expedient, as status information for example, to issue a current heat load and/or a duration of time for which the X-ray source can be operated for this examination.

Further output information can also be used in example embodiments to implement a teaching mode of operation in which users can be informed via appropriately displayed information about basic X-ray physics, functionalities of the X-ray source and such like.

Some of the functions of the status monitoring system described here can be specifically booked by the user, for example, by drawing up an appropriate service contract and suchlike. Then relevant enabling data can be stored, as a function of which output information relating to specific enabling services can be shown on the indicator device. Precisely in this context it is then advantageous if the at least one determination device(s) and/or the processing device(s) can have access to a wireless communications interface of the status monitoring system in order to be able to communicate with external facilities, for example with a control facility of the X-ray facility and/or an external server facility, in particular on the internet, be it to retrieve input information or even, however, to initiate output information and/or external functions, for example, specific after-sales services.

While it is conceivable in the context of the present invention to implement the status monitoring system, for example on a mobile device belonging to a user via a software application, that is, a computer program, which thus incorporates both the at least one determination device(s) and also the processing device(s), the touchscreen of the mobile device being used as an indicator device, for example, it is preferable according to the invention to equip the X-ray tube assembly itself with integrated intelligence.

A corresponding X-ray tube assembly 1 according to the invention is shown in more detail in its schematic structure in FIG. 2. The X-ray tube assembly 1 comprises an X-ray source 2, embodied here as an X-ray tube, to which in the present case acquisition device(s) 3, in particular sensors, are also assigned, for example to determine the temperature, the oil pressure, vibrations or such like. The X-ray tube assembly 1 can also include further acquisition device(s), in particular sensors, which measure ambient conditions as condition-related information, the humidity, for example.

Affixed on the X-ray tube assembly 1, a housing, for example, or at least pertaining to the X-ray tube assembly 1, an indicator device 5 with an assigned or integrated input device 6, which can be used to implement the method according to the invention, is further provided. So that the method according to the invention can be fully implemented by the X-ray tube assembly 1, said assembly further comprises a control unit 7 (such as at least one processor, for example), which incorporates both the at least one determination device(s) 8 and the processing device(s) 9. Using a wireless communications interface 10, such as a Bluetooth interface for example, communications links with external facilities can be established, only one control facility 11 of the X-ray facility and a server facility 12, in particular on the internet, being shown here. The control unit 7 can also communicate via an appropriate communications interface with mobile devices belonging to a user, for example, to use their indicator devices as further indicator devices, it being possible for other provision to be made to communicate with indicator devices wirelessly, for which indicator devices 5 installed on the X-ray tube assembly 1 and also NFC links can be used.

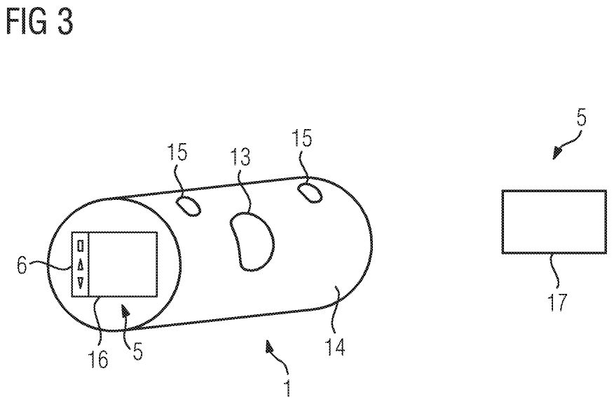

FIG. 3 shows a diagrammatic top view onto the X-ray tube assembly 1, in which the X-ray source is installed in a housing 14 such that only the outlet thereof 13 is visible, which housing also comprises appropriate affixing device(s) and/or mounting device(s) 15 for affixing the X-ray source in the X-ray facility. On a lateral surface the indicator device 5, which is embodied as a display 16, is now affixed with the assigned input device 6, which here comprises control elements. The display 16 can also be a touchscreen; however, for the implementation of the indicator device 5, an electronic paper display (EPD) that can use electronic ink is preferably provided. Such EPDs have a minimal power requirement or none at all when not in operation.

FIG. 3 also shows a further indicator device 5, which can be communicated with via the communications interface 10, an LCD display 17 for example, which can be arranged on a transportation pack or such like to implement the transportation monitoring that has already been described. Optionally, further indicator devices 5, which do not necessarily have to be part of the X-ray tube assembly 1 like the LCD display 17, can also be communicated with via the communications interface 10.

The embodiment of the X-ray tube assembly 1 that is described here makes it possible, moreover, to also use the display 16 to issue a machine-readable code thereon, in the present case a QR-Code, which directs to an external information source on the internet, for example, to a webpage with further useful information about the X-ray tube assembly 1 or the maintenance thereof. Thus, for example, in a simple manner, a user who is reading off the displayed machine-readable code, can be redirected to an external information source, on which, for example, further information about installations, maintenance and suchlike is available.

Although the invention has been illustrated and described in greater detail by the preferred embodiment, the invention is not restricted to the examples disclosed and other variants can be derived therefrom by a person skilled in the art without going beyond the scope of protection of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for monitoring the status of an X-ray tube assembly for an X-ray facility including an X-ray source, the method comprising:
determining input information, by at least one determination device, describing at least one of an application of the X-ray source, a current status of the X-ray source, and a current status of surroundings of the X-ray source;
deriving status information to be issued to a user, via at least one processing device, using the input information determined; and
displaying the status information derived on an indicator device, wherein condition-related information, describing transportation conditions during transportation, is used as the input information.

2. The method of claim 1, wherein the input information is at least one of
retrievable by at least one of a control facility of the X-ray facility, a control unit of the X-ray tube assembly, and an external server facility, and
acquirable by at least one acquisition device assigned or pertaining to the X-ray tube assembly.

3. The method of claim 1, wherein application-related information is used as input information, the application-related information at least one of describing a past application of the X-ray source, being usable for a directly impending measurement, being age-related information describing an age of the X-ray source, and being condition-related information describing storage conditions.

4. The method of claim 1, wherein a transportation procedure of the X-ray tube assembly is involved, and wherein transportation monitoring information is determined as status information.

5. The method of claim 4, wherein the determining of the condition-related information, describing transportation conditions, as the input information, includes comparing input information with target values for transportation, and wherein the status information derived is issued as a warning message upon the transportation conditions being outside a target range described by target values.

6. The method of claim 1, wherein prediction information describing a future usability of the X-ray source is the status information derived.

7. The method of claim 1, wherein the status information is issued as a function of a user input via an input device, at least one of
assigned to at least one of the indicator device, the X-ray tube assembly and the X-ray facility,
incorporated in one unit with the indicator device, and
issued as a function of a fulfillment of an output condition, evaluating at least one item of the input information.

8. The method of claim 7, wherein, at least one of in addition to a user input, subsequent to a user input and in fulfillment of a further output condition, at least one further item of output information, determined as relating to the X-ray source, is issued on the indicator device.

9. The method of claim 1, wherein the indicator device is affixed on the X-ray tube assembly or adjacent to the X-ray tube assembly.

10. The method of claim 9, wherein electronic paper or machine readable code, to direct the user to a different information source, is issued as at least one of the status information and further output information.

11. The method of claim 9, wherein at least part of the at least one determination device and the at least one processing device, forms a controller of the X-ray tube assembly.

12. The method of claim 1, wherein the indicator device is an indicator device of a mobile device of a user.

13. An X-ray tube assembly, comprising:
an X-ray source;
at least one processor, configured to determine input information, the input information describing at least one of an application of the X-ray source, a current status of the X-ray source, and surroundings of the X-ray source, and configured to derive status information to be issued to a user, utilizing the input information determined; and
an indicator device, controllable by the at least one processor, to display the status information derived, wherein condition-related information, describing transportation conditions during transportation, is used as the input information.

14. A non-transitory electronically readable data carrier storing a computer program, the computer program including program code configured to carry out the method of claim 1 when executed by a control facility.

15. A non-transitory electronically readable data carrier storing a computer program, the computer program including program code configured to carry out the method of claim 2 when executed by a control facility.

16. The method of claim 1, wherein the displaying of the status information derived is done on a display, as the indicator device.

17. The method of claim 2, wherein the input information is acquirable by a sensor.

18. The method of claim 2, wherein application-related information is used as input information, the application-related information at least one of describing a past application of the X-ray source, being usable for a directly impending measurement, being age-related information describing an age of the X-ray source, and being condition-related information describing storage conditions.

19. The method of claim 2, wherein a transportation procedure of the X-ray tube assembly is involved, and wherein transportation monitoring information is determined as status information.

20. The method of claim 19, wherein the determining of the condition-related information, describing transportation conditions, as the input information, includes comparing input information with target values for transportation, and wherein the status information derived is issued as a warning message upon the transportation conditions being outside a target range described by target values.
